(19) United States
(12) Patent Application Publication
(10) Pub. No.: US 2026/0216143 A1
(43) Pub. Date: Jul. 30, 2026

(54) PHARMACEUTICAL COMPOSITION FOR TREATING CHRONIC KIDNEY DISEASE

(71) Applicant: CURED Inc., Yokohama-shi (JP)

(72) Inventor: Masahiro MICHISHITA, Yokohama-shi (JP)

(73) Assignee: CURED Inc., Yokohama-shi (JP)

(21) Appl. No.: 19/578,109

(22) Filed: Mar. 25, 2026

Related U.S. Application Data
(62) Division of application No. 18/269,825, filed on Dec. 28, 2021.

(30) Foreign Application Priority Data
Dec. 28, 2020 (JP) 2020-219397

(51) Int. Cl. A61K 31/4184; A61K 31/202; A61K 31/41; A61P 13/12

(52) U.S. Cl. CPC A61K 31/4184; A61K 31/202; A61K 31/41; A61P 13/12

(57) ABSTRACT

For the object of establishing means for treating chronic kidney diseases including Alport syndrome, the present invention provides a pharmaceutical composition comprising a) a renin-angiotensin-system inhibitor and b) at least one type of an omega-3 polyunsaturated fatty acid, an ester thereof, a metabolite thereof, a pharmaceutically acceptable salt thereof, or a triglyceride thereof.

### CROSS-REFERENCE TO RELATED APPLICATIONS

This Application is a Divisional of co-pending U.S. patent application Ser. No. 18/269,825 filed on Jun. 27, 2023, which is the National Phase of PCT International Application No. PCT/JP2021/048741 filed on Dec. 28, 2021, which claims the priority benefit under 35 U.S.C. § 119 (a) to Japanese Patent Application No. 2020-219397 filed in Japan on Dec. 28, 2020, all of which are hereby expressly incorporated by reference into the present application.

### TECHNICAL FIELD

The present invention relates to a pharmaceutical composition used for the treatment of chronic kidney diseases including Alport syndrome.

### BACKGROUND ART

Alport syndrome is a rare, hereditary disease in which progressive nephritis occurs inevitably in early life because of a mutation in the type IV collagen gene, and the disease is sometimes accompanied by hearing impairment, eye lesions, and diffuse leiomyoma. Since the disease leads to terminal renal failure at a young age, it is designated as an intractable disease by the national government in Japan.

In type IV collagen, α-chains form triple helixes, and these polymers form a network structure. In Alport syndrome, impaired syntheses of any or all of type IV collagen α3, α4, and α5 chain proteins result in failure to form the mesh structure of collagen. The glomerular filtration membrane is formed by vascular endothelial cells, the basement membrane, and foot processes of glomerular epithelial cells. In the glomerulus in Alport syndrome, incomplete formation or completely deficiency of type IV collagen constituting the basement membrane causes an abnormal glomerular filtration function and chronic renal impairment associated therewith.

A mouse having homozygous deficiency in the type IV collagen α3 chain gene (Col4a3−/− mouse) is a model of an autosomal recessive clinical condition of Alport syndrome. Because of the small variation in the clinical condition, the average life span is almost consistent. Specifically, renal tubular expansion, inflammatory cell infiltration, crescent formation, and fibrosis begin to occur, and the serum creatinine concentration rapidly increases from six weeks old, some deaths from terminal renal failure begin to occur at eight weeks old or older, and the majority of animals die at the age of 10 weeks old. Given such characteristics, this mouse model is widely used worldwide because the efficacy of a drug can be assessed in a short period.

A nonclinical study using the Col4a3−/− mice has reported that the survival time significantly prolonged, and the urine protein level and the serum urea concentration markedly decreased in a group treated with ramipril (an angiotensin converting enzyme (ACE) inhibitor) or candesartan (an angiotensin II receptor antagonist), which are types of renin-angiotensin (RA)-system inhibitors used as standard therapeutic agents for hypertension (Non Patent Literature 1). A clinical study, a retrospective analysis by Gross et al. on the relationship between oral administration of a renin-angiotensin-system inhibitor and renal impairment in 283 patients with Alport syndrome, also demonstrated an effect of significantly delaying the median age of introduction of a renal replacement therapy in a group receiving oral administration of a renin-angiotensin-system inhibitor (Non Patent Literature 2). The Clinical Practice Guideline for Alport Syndrome 2017 (The Japanese Society for Pediatric Nephrology) in Japan also recommends administering renin-angiotensin-system inhibitors to patients with Alport syndrome in order to suppress progression of renal impairment.

### CITATION LIST

### Non Patent Literature

[Non Patent Literature 1] Oliver Gross et al., Nephrol Dial Transplant (2004) 1716-23
[Non Patent Literature 2] Oliver Gross et al., Kidney Int (2012) 81:494-501

### SUMMARY OF INVENTION

### Technical Problem

While renin-angiotensin-system inhibitors are administered to patients with Alport syndrome as described above, development of a drug for treating Alport syndrome more effectively has been awaited. The present invention was made under such a background, and an object of the present invention is to provide means for treating chronic kidney diseases including Alport syndrome.

### Solution to Problem

The present inventors studied assiduously to solve the above-mentioned problem. As a result, they found that the survival rate was markedly improved in Alport syndrome model mice by administering an angiotensin II receptor antagonist and an omega-3 polyunsaturated fatty acid ethyl ester to these mice and thus accomplished the present invention.

That is, the present invention provides the following (1) to (15):
(1) A pharmaceutical composition comprising a) a renin-angiotensin-system inhibitor and b) at least one type of an omega-3 polyunsaturated fatty acid, an ester thereof, a metabolite thereof, a pharmaceutically acceptable salt thereof, or a triglyceride thereof.
(2) A pharmaceutical composition for use in combination with a renin-angiotensin-system inhibitor, the pharmaceutical composition comprising at least one type of an omega-3 polyunsaturated fatty acid, an ester thereof, a metabolite thereof, a pharmaceutically acceptable salt thereof, or a triglyceride thereof.
(3) A pharmaceutical composition for use in combination with an omega-3 polyunsaturated fatty acid, an ester thereof, a metabolite thereof, a pharmaceutically acceptable salt thereof, or a triglyceride thereof, the pharmaceutical composition comprising a renin-angiotensin-system inhibitor.
(4) The pharmaceutical composition according to any of (1) to (3), which is used for the treatment of a chronic kidney disease.
(5) The pharmaceutical composition according to (4), wherein the chronic kidney disease is a kidney disease with glomerular damage.
(6) The pharmaceutical composition according to (4), wherein the chronic kidney disease is a kidney disease with podocyte damage or a kidney disease with glomerular basement membrane damage.
(7) The pharmaceutical composition according to (4), wherein the chronic kidney disease is a tubular kidney disease or an interstitial kidney disease.
(8) The pharmaceutical composition according to (4), wherein the chronic kidney disease is Alport syndrome.
(9) The pharmaceutical composition according to any of (1) to (8), wherein the renin-angiotensin-system inhibitor is an angiotensin II receptor antagonist.
(10) The pharmaceutical composition according to any of (1) to (8), wherein the renin-angiotensin-system inhibitor is candesartan cilexetil.
(11) The pharmaceutical composition according to any of (1) to (8), wherein the renin-angiotensin-system inhibitor is valsartan.
(12) The pharmaceutical composition according to any of (1) to (11), wherein the at least one type of an omega-3 polyunsaturated fatty acid is eicosapentaenoic acid, docosahexaenoic acid, or eicosapentaenoic acid and docosahexaenoic acid.
(13) The pharmaceutical composition according to any of (1) to (11), wherein the at least one type of an omega-3 polyunsaturated fatty acid is eicosapentaenoic acid and docosahexaenoic acid.
(14) The pharmaceutical composition according to any of (1) to (11), wherein the at least one type of an omega-3 polyunsaturated fatty acid is docosahexaenoic acid.
(15) The pharmaceutical composition according to any of (1) to (11), wherein the at least one type of an omega-3 polyunsaturated fatty acid is eicosapentaenoic acid.

The present specification encompasses the content disclosed in the specification and/or the drawings of Japanese Patent Application No. 2020-219397, which is the basis of the priority of the present application.

### Advantageous Effects of Invention

The present invention provides a novel pharmaceutical composition. This pharmaceutical composition can be used for the treatment of chronic kidney diseases including Alport syndrome.

### BRIEF DESCRIPTION OF DRAWINGS

FIG. 1 shows changes with time in the survival rate in each group of Alport syndrome model mice. In the figure, PBS denotes the PBS group (an untreated group), CAND denotes the candesartan monotherapy group, PFD denotes the Pirespa monotherapy group, LOT denotes the Lotriga monotherapy group, CAND+PFD denotes the candesartan and Pirespa combination therapy group, and CAND+LOT denotes the candesartan and Lotriga combination therapy group.

FIG. 2 shows changes with time in the mean body weight in each group of Alport syndrome model mice. In the figure, WT denotes wild-type mice, PBS denotes the PBS group (an untreated group), CAND denotes the candesartan monotherapy group, PFD denotes the Pirespa monotherapy group, LOT denotes the Lotriga monotherapy group, CAND+PFD denotes the candesartan and Pirespa combination therapy group, and CAND+LOT denotes the candesartan and Lotriga combination therapy group.

FIG. 3 shows changes with time in the sum of body weights in each group of Alport syndrome model mice. In the figure, WT denotes wild-type mice, PBS denotes the PBS group (an untreated group), CAND denotes the candesartan monotherapy group, PFD denotes the Pirespa monotherapy group, LOT denotes the Lotriga monotherapy group, CAND+PFD denotes the candesartan and Pirespa combination therapy group, and CAND+LOT denotes the candesartan and Lotriga combination therapy group.

FIG. 4 shows the results of blood biochemistry tests (kidney-related items) in each group of Alport syndrome model mice. Each bar in the graph represents, from the far left, wild-type mice (WT), the PBS group (PBS), the candesartan monotherapy group (CAND), the Pirespa monotherapy group (PFD), the Lotriga monotherapy group (LOT), the candesartan and Pirespa combination therapy group (CAND+PFD), or the candesartan and Lotriga combination therapy group (CAND+LOT).

FIG. 5 shows the results of tests of bone mineral metabolism-related items in each group of Alport syndrome model mice. Each bar in the graph represents, from the far left, wild-type mice (WT), the PBS group (PBS), the candesartan monotherapy group (CAND), the Pirespa monotherapy group (PFD), the Lotriga monotherapy group (LOT), the candesartan and Pirespa combination therapy group (CAND+PFD), or the candesartan and Lotriga combination therapy group (CAND+LOT).

FIG. 6 shows the results of blood biochemistry tests (pancreas and liver-related items) in each group of Alport syndrome model mice. Each bar in the graph represents, from the far left, wild-type mice (WT), the PBS group (PBS), the candesartan monotherapy group (CAND), the Pirespa monotherapy group (PFD), the Lotriga monotherapy group (LOT), the candesartan and Pirespa combination therapy group (CAND+PFD), or the candesartan and Lotriga combination therapy group (CAND+LOT).

FIG. 7 shows the HE-staining images of the kidneys of wild-type mice (WT) and the PBS group (an untreated group), the candesartan monotherapy group (CAND), and the candesartan and Lotriga combination therapy group (CAND+LOT) of Alport syndrome model mice.

FIG. 8 shows the PAS-staining images of the kidneys of wild-type mice (WT) and the PBS group (an untreated group), the candesartan monotherapy group (CAND), and the candesartan and Lotriga combination therapy group (CAND+LOT) of Alport syndrome model mice.

FIG. 9 shows the MT-staining images of the kidneys of wild-type mice (WT) and the PBS group (an untreated group), the candesartan monotherapy group (CAND), and the candesartan and Lotriga combination therapy group (CAND+LOT) of Alport syndrome model mice.

FIG. 10 shows the PAM-staining images of the kidneys of wild-type mice (WT) and the PBS group (an untreated group), the candesartan monotherapy group (CAND), and the candesartan and Lotriga combination therapy group (CAND+LOT) of Alport syndrome model mice.

FIG. 11 shows the severity of glomerular damage based on the pathology image of the kidneys in each group of Alport syndrome model mice. In the figure, WT denotes wild-type mice, PBS denotes the PBS group (an untreated group), CAND denotes the candesartan monotherapy group, PFD denotes the Pirespa monotherapy group, LOT denotes the Lotriga monotherapy group, CAND+PFD denotes the candesartan and Pirespa combination therapy group, and CAND+LOT denotes the candesartan and Lotriga combination therapy group.

FIG. 12 shows the severity of renal tubular and interstitial damage based on the pathology image of the kidneys in each group of Alport syndrome model mice. In the figure, WT denotes wild-type mice, PBS denotes the PBS group (an untreated group), CAND denotes the candesartan monotherapy group, PFD denotes the Pirespa monotherapy group, LOT denotes the Lotriga monotherapy group, CAND+PFD denotes the candesartan and Pirespa combination therapy group, and CAND+LOT denotes the candesartan and Lotriga combination therapy group.

FIG. 13 shows changes with time in the survival rate in the candesartan monotherapy group and the omega-3 polyunsaturated fatty acid combination therapy groups.

FIG. 14 shows changes with time in the survival rate in the combination therapy groups of candesartan and a docosahexaenoic acid ethyl ester or an eicosapentaenoic acid ethyl ester.

FIG. 15 shows changes with time in the mean body weight in the combination therapy groups of candesartan and a docosahexaenoic acid ethyl ester or an eicosapentaenoic acid ethyl ester. ** p<0.01: A significant difference from the untreated group was detected by t test. When the mean body weight was calculated, the body weight of a dead mouse was not assumed as zero, and the body weight measured immediately before death was used for calculation.

FIG. 16 shows changes with time in the mean body weight in each study group. ** p<0.01: A significant difference from the untreated group was detected by t test.

FIG. 17 shows the percent change in the mean body weight and the survival rate at the end of the study in each group.

FIG. 18 shows changes with time in the survival rate in each study group.

FIG. 19 shows the percent change in the mean body weight and the survival rate at the end of the study in each group.

### DESCRIPTION OF EMBODIMENTS

The present invention will be described in detail below.

The pharmaceutical composition of the present invention is a pharmaceutical composition for administering a) a renin-angiotensin-system inhibitor and b) at least one type of an omega-3 polyunsaturated fatty acid, an ester thereof, a metabolite thereof, a pharmaceutically acceptable salt thereof, or a triglyceride thereof to a patient. It is sufficient that the pharmaceutical composition of the present invention can administer the above-mentioned a) and the above-mentioned b) to a patient. For example, the pharmaceutical composition of the present invention may be a pharmaceutical composition comprising the above-mentioned a) and the above-mentioned b), a pharmaceutical composition comprising the above-mentioned b) for use in combination with the above-mentioned a), or a pharmaceutical composition comprising the above-mentioned a) for use in combination with the above-mentioned b).

The renin-angiotensin-system inhibitor to be used may be an angiotensin converting enzyme inhibitor, an angiotensin II receptor antagonist, or a renin inhibitor, and is preferably an angiotensin II receptor antagonist, more preferably candesartan cilexetil or valsartan, yet more preferably candesartan cilexetil. Of note, the above-mentioned “renin-angiotensin-system inhibitor,” “angiotensin converting enzyme inhibitor,” “angiotensin II receptor antagonist,” and “renin inhibitor” also include substances (so-called prodrugs) which undergo metabolism such as oxidation, reduction, or hydrolysis in an organism and serve as a renin-angiotensin-system inhibitor, an angiotensin converting enzyme inhibitor, an angiotensin II receptor antagonist, or a renin inhibitor.

Known angiotensin converting enzyme inhibitors can be used, and examples thereof include captopril, cilazapril, enalapril, fosinopril, lisinopril, quinapril, ramipril, zofenopril, imidapril, temocapril, perindopril, alacepril, delapril, benazepril, and trandolapril. Known angiotensin II receptor antagonists can also be used, and examples thereof include candesartan, candesartan cilexetil, eprosartan, irbesartan, losartan, tasosartan, telmisartan, valsartan, azilsartan, and olmesartan.

Known omega-3 polyunsaturated fatty acids can also be used, and examples thereof include eicosapentaenoic acid, docosahexaenoic acid, and α-linolenic acid. The pharmaceutical composition of the present invention may contain only one type of an omega-3 polyunsaturated fatty acid, for example, only eicosapentaenoic acid or only docosahexaenoic acid, or may contain two or more types of omega-3 polyunsaturated fatty acids, for example, eicosapentaenoic acid and docosahexaenoic acid.

Instead of an omega-3 polyunsaturated fatty acid, an ester thereof may be used. Specific examples of the ester include a methyl ester, an ethyl ester, a propyl ester, and an ester bound with a phospholipid or a lysophospholipid. Preferred examples of the omega-3 polyunsaturated fatty acid ester include ethyl eicosapentaenoate and ethyl docosahexaenoate. Further, the pharmaceutical composition of the present invention may contain a metabolite of an omega-3 polyunsaturated product, for example, resolvin D1 to D4, resolvin E1 to E2, protectin D1, 17S-HDHA, a metabolite analogue thereof, or the like. Further, instead of an omega-3 polyunsaturated fatty acid, the pharmaceutical composition of the present invention may also contain a derivative such as a pharmaceutically acceptable salt thereof or a triglyceride thereof.

An omega-3 polyunsaturated fatty acid, an ester thereof, a metabolite thereof, a pharmaceutically acceptable salt thereof, or a triglyceride thereof is preferably 1) docosahexaenoic acid, an ester thereof, a metabolite thereof, a pharmaceutically acceptable salt thereof, or a triglyceride thereof, or 2) eicosapentaenoic acid, an ester thereof, a metabolite thereof, a pharmaceutically acceptable salt thereof, or a triglyceride thereof, more preferably docosahexaenoic acid, an ester thereof, a metabolite thereof, a pharmaceutically acceptable salt thereof, or a triglyceride thereof, yet more preferably an ester of docosahexaenoic acid, particularly preferably docosahexaenoic acid ethyl ester.

The weight ratio of an angiotensin converting enzyme inhibitor and an omega-3 polyunsaturated fatty acid in the pharmaceutical composition is not particularly limited, and the ratio of an omega-3 polyunsaturated fatty acid to an angiotensin converting enzyme inhibitor in the pharmaceutical composition can be made, for example, 150 to 2000:1, preferably 150 to 1000:1, more preferably 300 to 1000:1.

The pharmaceutical composition of the present invention can be used for the treatment of a chronic kidney disease. The term “treatment” used herein means not only suppression of progression of a chronic kidney disease and complete cure of a chronic kidney disease, but also prevention of a chronic kidney disease.

The term “chronic kidney disease” used in the present invention means a condition in which the kidney function is reduced as compared with a healthy subject (for example, a condition in which the glomerular filtration rate has been reduced to lower than 60 mL/min/1.73 m²) or a condition in which abnormalities of the kidneys such as occurrence of proteinuria are persistent. The following kidney diseases are included in the “chronic kidney disease” of the present invention: Alport syndrome, focal segmental glomerulosclerosis, minimal change nephrotic syndrome, membranous nephropathy, HIV-1-associated nephropathy, diffuse mesangial sclerosis, congenital nephrosis syndrome of the Finnish type, lupus nephritis, collapsing glomerulopathy, diabetic nephropathy, hypertensive nephrosclerosis, obesity-related glomerulopathy, IgA nephropathy, polycystic kidney disease (ADPKD, ARPKD), mesangial proliferative glomerulonephritis, Epstein syndrome, nail-patella syndrome (osteoonychodysplasia), fibronectin nephropathy, lipoprotein glomerulopathy, chronic pyelonephritis, familial juvenile hyperuricemic nephropathy, hereditary nephrosis syndrome, and tubulointerstitial nephritis. Further, the following diseases are classified into rapidly progressive glomerulonephritis syndromes (RPGNs), but since the reduced renal function persists after treatment and the diseases often progress to chronic kidney diseases, they are included in the “chronic kidney diseases” in the present invention: crescentic nephritis, ANCA-associated glomerulonephritis, anti-GBM antibody glomerulonephritis, purpura nephritis, and cryoglo-bulinemic nephritis. The expression “suppression of progression of a chronic kidney disease” refers to suppression of, for example, glomerular damage or renal tubular damage. Examples of the glomerular damage include glomerulosclerosis, crescent formation, and basement membrane with a double contour. Examples of the renal tubular damage include renal tubular expansion accompanied by hyaline casts, interstitial fibrosis, inflammatory cell infiltration into the interstitium, and renal tubular expansion accompanied by cellular casts.

It is sufficient that the treatment target is a chronic kidney disease, but the treatment target is preferably a kidney disease with glomerular damage, a tubular kidney disease, or a kidney disease with interstitial damage, more preferably a kidney disease with podocyte damage or a kidney disease with glomerular basement membrane damage, yet more preferably Alport syndrome.

The pharmaceutical composition of present invention is usually administered orally but may be administered through other routes. For example, the pharmaceutical composition of present invention may be administered through a sublingual, intracutaneous, or subcutaneous route or through the muscle, the peripheral or central vein, the artery, the lymphatic vessel, the anus, the nasal cavity, the respiratory tract, or the peritoneal cavity.

An active ingredient and other components such as a nutrient and an excipient can be added to the pharmaceutical composition of the present invention as required. Examples of the dosage form of the pharmaceutical composition include tablet, capsule, subtilized granule, syrup, injection, drip infusion, poultice, and suppository. These dosage forms can be formulated by usual methods suitably using a solvent, a dispersion medium, an extender, an excipient, or the like.

The pharmaceutical composition of the present invention may be in any form as long as it is in a form in which both a) a renin-angiotensin-system inhibitor and b) at least one type of an omega-3 polyunsaturated fatty acid, an ester thereof, a metabolite thereof, a pharmaceutically acceptable salt thereof, or a triglyceride thereof can be administered to a patient. For example, both the above-mentioned a) and the above-mentioned b) may be formulated in one form (a combination drug), or each of the above-mentioned a) and the above-mentioned b) may be formulated in separate dosage forms (combined formulations). When they are formulated in separate dosage forms, these formulations may be administered simultaneously or administered separately with a time difference. Further, when the formulations are administered with a time difference, the above-mentioned a) may be administered first followed by administration of the above-mentioned b), or the above-mentioned b) may be administered first followed by administration of the above-mentioned a). The administration methods for the formulations may be the same or different.

The dose of the pharmaceutical composition of the present invention is not particularly limited, but a renin-angiotensin-system inhibitor, which is one of the active ingredients, is preferably administered to an adult at a dose of 2 to 12 mg daily, more preferably 4 to 8 mg daily.

### EXAMPLES

The present invention will be described in more detail with the following examples, but the scope of the present invention is not in any way limited to these examples.

### Example 1

i. Subcontracting of the Study

The following study was subcontracted to an external facility.

### 2. Objective

Each test substance is orally and repeatedly administered to Alport syndrome model (129-Col4a3tm1Dec/J) mice for 10 weeks, and then a serum biochemistry test is performed. The kidneys are further observed histopathologically to examine the effect of administration of each test substance on the kidneys.

### 3. Animal Welfare

All animal experiments performed in this example were planned in accordance with the laboratory animal welfare rules of the subcontractor and performed after approval by the laboratory animal welfare committee. Of note, the conducted animal study complied with the “Basic guidelines for the implementation of animal experiments etc. at research laboratories under jurisdiction of the Ministry of Agriculture, Forestry and Fisheries” (2006 Agriculture, Forestry and Fisheries Research Council Office Notification) and the like.

### 4. Study Materials

### 4.1. Test Substances (Known Compounds)

### 4.1.1. Names

TABLE 1

| Name | General description | Storage condition |
|---|---|---|
| Candesartan | Powder | Refrigerated (0° C.-10° C.) |
| Pirespa | Powder | Room temperature (15° C.-25° C.) |
| Lotriga | Granular capsule | Room temperature (15° C.-25° C.) |

Candesartan is a therapeutic agent for hypertension which is classified as an angiotensin II receptor antagonist among renin-angiotensin-system inhibitors. The renoprotective effect thereof is known.

Pirespa is a therapeutic agent for idiopathic pulmonary fibrosis which has an anti-fibrosis effect and suppresses fibrosis of the lung interstitium.

Lotriga is a therapeutic agent for hyperlipidemia which is an omega-3 polyunsaturated fatty acid ethyl formulation and contains ethyl eicosapentaenoate and ethyl docosahexaenoate as principal components.

### 4.1.2. Method for Preparing Test Substances Etc.

Test substances were prepared for each use before use on the experiment day. Candesartan was dissolved at a dose of 4 mg/mL in phosphate-buffered saline (hereinafter referred to as PBS) to prepare a dosing solution (10 mg/2.5 mL/kg) for Group Numbers 6 and 7. A solution was prepared by diluting this dosing solution with an equal amount of PBS as a dosing solution (10 mg/5 ml/kg) for Group Number 3. Pirespa was dissolved at a dose of 40 mg/mL in PBS and dissolved in PBS which was to be a dosing solution (50 mg/1.25 mL/kg) for Group Number 6. A solution was prepared by diluting this dosing solution with an equal amount of PBS as a dosing solution (50 mg/2.5 mL/kg) for Group Number 4. Lotriga was suspended at a dose of 600 mg/mL in a 0.5% methylcellulose solution (hereinafter referred to as MC) to prepare a dosing solution (1500 mg/2.5 mL/kg) for Group Number 7. A solution was prepared by diluting this dosing solution with an equal amount of MC as a dosing solution (1500 mg/5 mL/kg) for Group Number 5.

### 4.2. Animal Experiment

### 4.2.1. Animals Used

Male 129-Col4a3tm1Dec/J mice (hereinafter referred to as Homo mice) were introduced into the study facility at three weeks old, and healthy animals (four weeks old), which did not show any abnormality in general signs during the acclimatization period of about one week, were subjected to the experiment.

### 4.2.2. Breeding Environment

Up to five animals were housed in a breeding cage (189 mm×298 mm×128 mm) and bred in an animal breeding room adjusted to the following environment: temperature, 20° C. to 25° C.; humidity, 40% to 70%; ventilation rate, 10 times or more/hour; light on, 12 hours (7:00 to 19:00). Animals were given solid feed CRF-1 (Oriental Yeast Co., Ltd.) ad libitum. As a drinking water, water filtered with a filter was given ad libitum.

### 5. Experimental Methods

### 5.1. Group Composition

TABLE 2

| Group | | No. of | Substance | Dose | Volume atministered | Administration |
|---|---|---|---|---|---|---|
| Number | Mouse | animals | administered | (mg/kg) | (mL/kg) | condition |
| 1 | WT | 4 | Vehicle | — | 5 | Once daily |
| 2 | AS | 5 | Vehicle | — | 5 | Once daily |
| 3 | AS | 5 | Candesartan | 10 | 5 | Once daily |
| 4 | AS | 5 | Pirespa | 100 (50 | 2.5 | Twice daily |
| | | | | per dose) | | (at a >6-hour interval) |
| 5 | AS | 5 | Lotriga | 1500 | 5 | Once daily |
| 6 | AS | 5 | Candesartan | 10 | 2.5 | Once daily |
| | | | Pirespa | 100 (50 | 1.25 | Twice daily |
| | | | | per dose) | | (at a >8-hour interval) |
| 7 | AS | 5 | Candesartan | 10 | 2.5 | Once daily |
| | | | Lotriga | 1500 | 2.5 | Once daily |

### 5.2. Grouping

Animals identified as Homo mice (AS in Table 2) by a genotyping test were grouped into Group Numbers 2 to 7 on the basis of the body weight at four weeks old, so that the mean body weight should be close between groups. Four animals were extracted from Group Number 1, so that the body weight should be close to those of the Homo mice groups. After grouping, individual animals were identified using ear tags. Animals excluded from the study and animals found to have abnormality during the acclimatization period were euthanized.

### 5.3. Observation of General Signs

General signs were observed once daily from the experiment start day (grouping day) to the experiment completion day (sampling day).

### 5.4. Measurement of Body Weight

Body weight was measured using a scale for animals twice weekly from the grouping day and on the sampling day of each group.

### 5.5. Administration of Test Substances

Animals were restrained, and a test substance was orally administered using a 1-mL syringe and a sonde. Administration was performed in accordance with the conditions described in Section 5.1., so that a volume of approximately 0.1 mL should be administered to each animal. The volume administered was calculated on the basis of the most recent body weight. Pirespa was administered at a daily dose of 100 mg/kg which was divided into two doses. The administration interval was 6 hours or longer.

### 5.6. Collection of Biomaterials

The body weights of animals were measured at Day 45, blood was drawn from the caudal vena cava under anesthesia with isoflurane, and animals were euthanized by exsanguination. Death was confirmed by visually observing cardiopulmonary arrest (euthanasia). Then, the left and right kidneys, the left and right femurs, the left and right eyeballs, the cochlea, the heart, and the pancreas were collected and trimmed, and the wet weights of the left and right kidneys, the left and right femurs, and the heart were measured. Among the collected biotissues, the left and right femurs were immersed in a 70% ethanol solution. Other tissues were immersed in a 10% neutral buffered formalin solution. Of note, the right kidney was divided into two at the renal pelvis and immersed after to obtain kidney specimens.

### 5.7. Blood Biochemistry Test

### 5.7.1. Preparation of Serum

The obtained blood was allowed to stand at room temperature for at least four hours and centrifuged under conditions of 4° C., 12000 rpm, and 3 min to obtain serum. Serum was divided into two tubes and frozen.

### 5.7.2. Measurement of Each Item

Blood biochemistry test items were measured using the frozen serum delegated to the external facility. The measurement items were total protein, albumin, blood urea nitrogen (BUN), creatinine (CRE), uric acid, sodium (Na), potassium (K), chloride (CI), calcium, phosphorus, amylase, lipase, AST (GOT), ALT (GPT), Y-GTP, LDH, triglyceride, total cholesterol, HDL cholesterol, total bilirubin, and glucose. Pooled specimens of each group were used for measurement.

### 5.8. Histopathological Test

The kidneys immersed in a 10% neutral buffered formalin solution were sent to the external facility, and Hematoxylin-Eosin (HE) staining, Periodic Acid-Schiff (PAS) staining, Masson's Trichrome (MT) staining, and Periodic Acid Methenamine silver (PAM) staining were performed for pathological assessment.

### 6. Data Analysis

The mean and standard error of body weight and weights of the kidney and femur tissue were calculated for each study group.

### 7. Humanitarian Endpoints

Euthanasia treatment was performed for animals excluded from the study as a result of grouping or animals which developed such aggravated conditions as listed below or a refractory condition. Euthanasia treatment was performed by a carbon dioxide inhalation method.

Rapid body weight loss: loss of 25% or more in seven days

Distress symptoms: dyspnea, piloerection, crouching position, extreme cyanosis

Long-term abnormalities in appearance with no sign of recovering: inability to change the body position, diarrhea, hemorrhage, vulvar stains

### 8. Experimental Results

8.1. Changes with Time in the Survival Rate

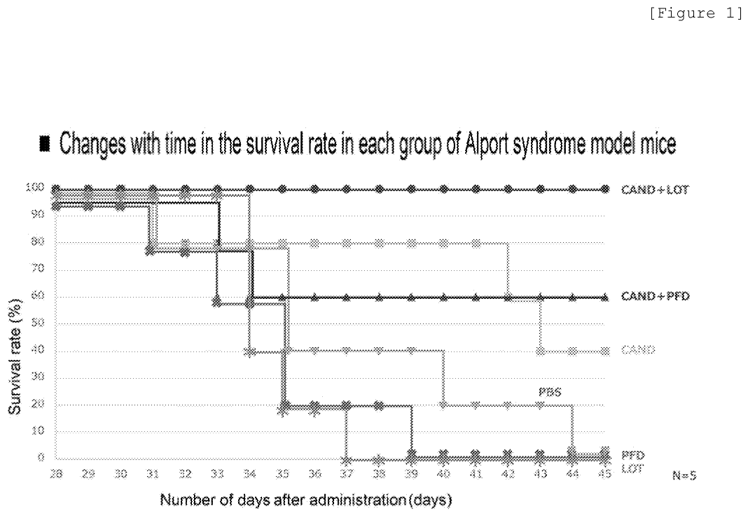

FIG. 1 shows changes with time in the survival rate in each group of Alport syndrome model mice.

The study was completed at Day 45, which is the following day of confirmation of deaths of all animals in the PBS group (an untreated group). Although all mice died early in the Lotriga monotherapy group and the Pirespa monotherapy group, all mice survived with a favorable general condition in the candesartan and Lotriga combination therapy group. The survival rate in the candesartan and Pirespa combination therapy group was 60%, which was higher than the survival rate (40%) in the candesartan monotherapy group.

8.2. Changes with Time in the Mean Body Weight

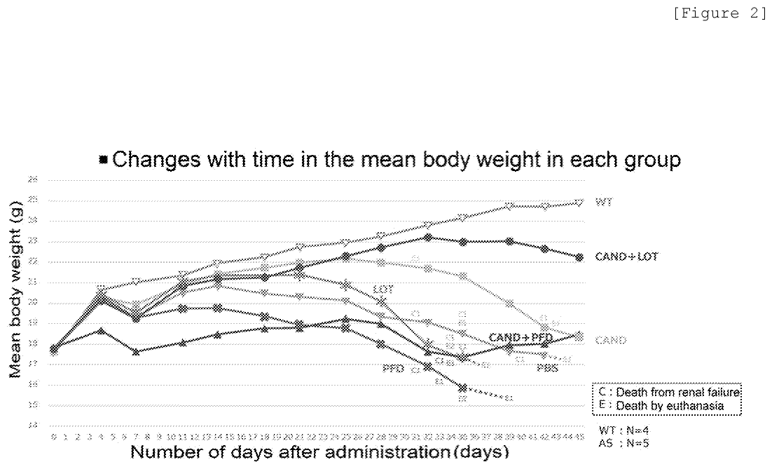

FIG. 2 shows changes with time in the mean body weight in each group of Alport syndrome model mice. Of note, when the mean body weight was calculated, the body weight of a dead mouse was not assumed as zero, and the body weight measured immediately before death was used for calculation.

In the candesartan and Lotriga combination therapy group, the mean body weight increased by as much as in wild-type mice until 32 days after administration. Although the body weight decreased slightly thereafter until the end of the experiment, the body size was clearly larger as compared with other groups, and the general condition was favorable. In the candesartan monotherapy group, body weight loss began from 25 days after administration, and marked body weight loss was also observed in surviving mice.

8.3. Changes with Time in the Sum of Body Weights

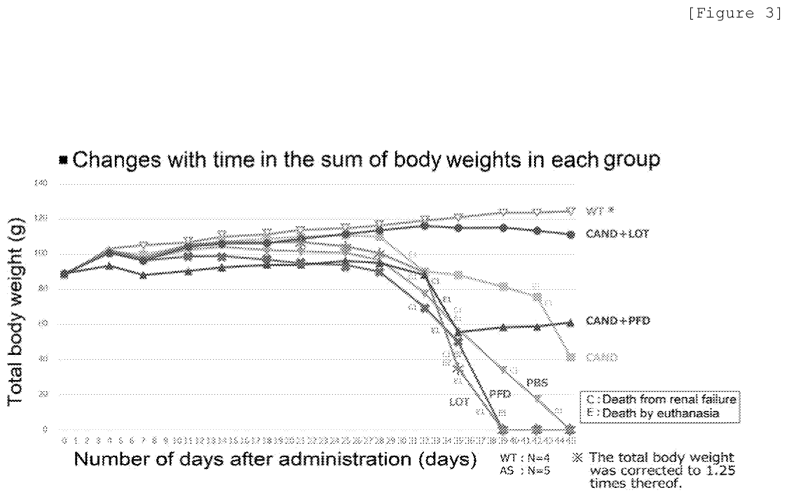

FIG. 3 shows changes with time in the sum of body weights in each group of Alport syndrome model mice.

When the sum of body weights of the mice surviving at the time of body weight measurement was plotted with time, the difference between the candesartan and Lotriga combination therapy group and the candesartan monotherapy group became clearer. The sum in the candesartan and Pirespa combination therapy group eventually became higher than that in the candesartan monotherapy group but was not as high as that in the candesartan and Lotriga combination therapy group.

### 8.4. Blood Biochemistry Tests (Kidney-Related Items)

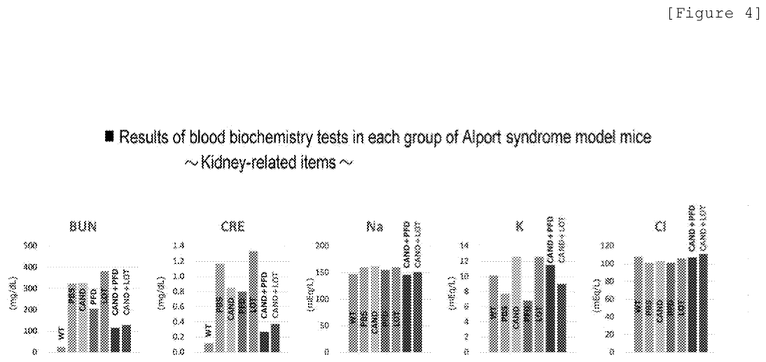

FIG. 4 shows the results of the blood biochemistry tests (kidney-related items) in each group of Alport syndrome model mice.

In the candesartan and Lotriga combination therapy group (survival rate, 100%) and the candesartan and Pirespa combination therapy group (survival rate, 60%), the BUN and creatinine levels were higher than the upper limits of normal ranges, but much lower than in the untreated group and the candesartan monotherapy group, and the Na, K, and Cl levels were all within the normal ranges. In the candesartan monotherapy group (survival rate, 40%), the BUN and creatinine levels were very high and as high as in the untreated group (survival rate, 0%), indicating development of hyperkalemia. While progression of renal failure was clearly delayed in the candesartan and Lotriga combination therapy group, renal failure progressed in the candesartan monotherapy group at a level comparable to that in the untreated group.

### 8.5. Tests of Bone Mineral Metabolism-Related Items

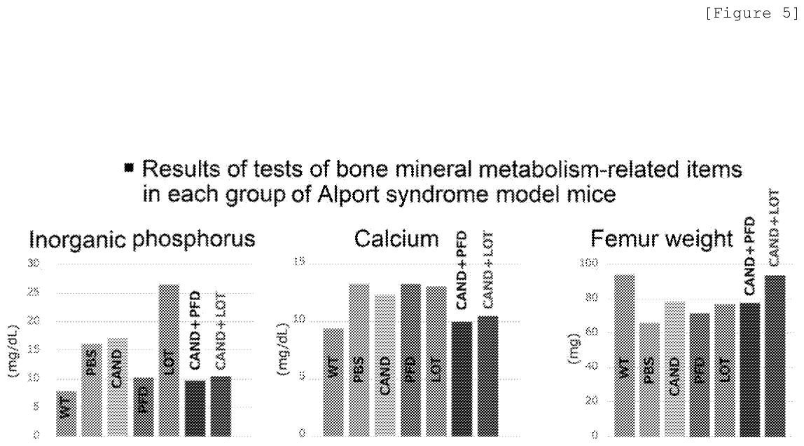

FIG. 5 shows the results of tests of bone mineral metabolism-related items in each group of Alport syndrome model mice.

In the candesartan and Lotriga combination therapy group (survival rate, 100%), the serum calcium and inorganic phosphorus levels remained within the normal ranges, and the femur weight remained at a level comparable to that in wild-type mice. It is considered that progression of renal failure was protracted, but it did not lead to renal osteodystrophy, and ectopic calcification did not occur in either blood vessels or key organs, which contributed to improvement of the survival rate greatly.

### 8.6. Blood Biochemistry Tests (Pancreas and Liver-Related Items)

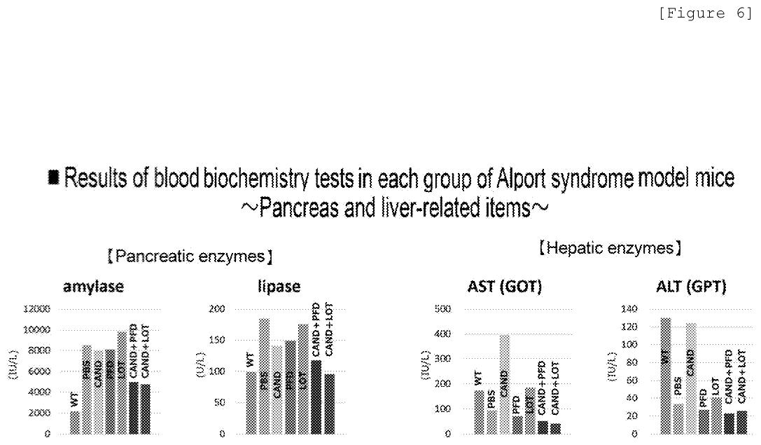

FIG. 6 shows the results of the blood biochemistry tests (pancreas and liver-related items) in each group of Alport syndrome model mice.

In the candesartan and Lotriga combination therapy group (survival rate, 100%), the amylase levels were much lower than in the untreated group and the candesartan monotherapy group, and the lipase levels were normal. Therefore, combination use of candesartan and Lotriga is considered to have prevented development of secondary pancreatitis by protracted progression of renal failure. In the candesartan and Lotriga combination therapy group, the effect on liver function remained within the normal range for both AST and ALT.

### 8.7. Histopathological Test of the Kidneys (HE Staining)

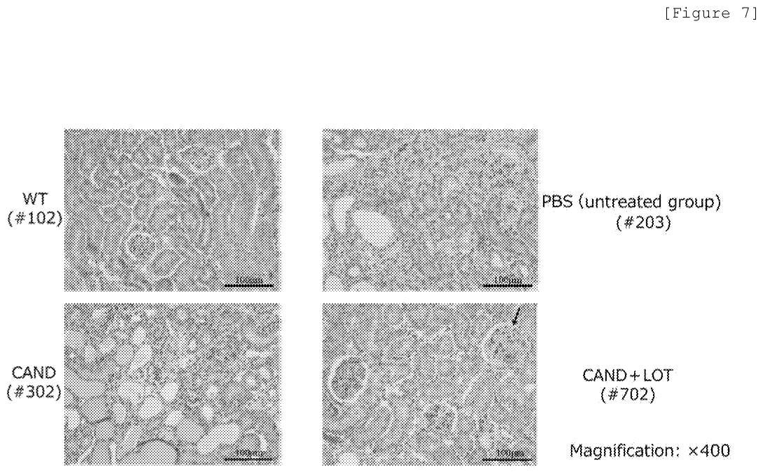

FIG. 7 shows the HE-staining images of the kidneys in wild-type mice, the PBS group, the candesartan monotherapy group, and the candesartan and Lotriga combination therapy group.

In the PBS group and the candesartan monotherapy group, adhesion to the glomerulus due to glomerulosclerosis and crescent formation, renal tubular expansion, and lymphocyte infiltration into the interstitium were observed at various sites. In the candesartan and Lotriga combination therapy group, however, glomerulosclerosis and crescent formation were rarely observed, and no change was observed in either the renal tubules or the interstitium. The arrow shows an example of the normal glomerulus.

### 8.8. Histopathological Test of the Kidneys (PAS Staining)

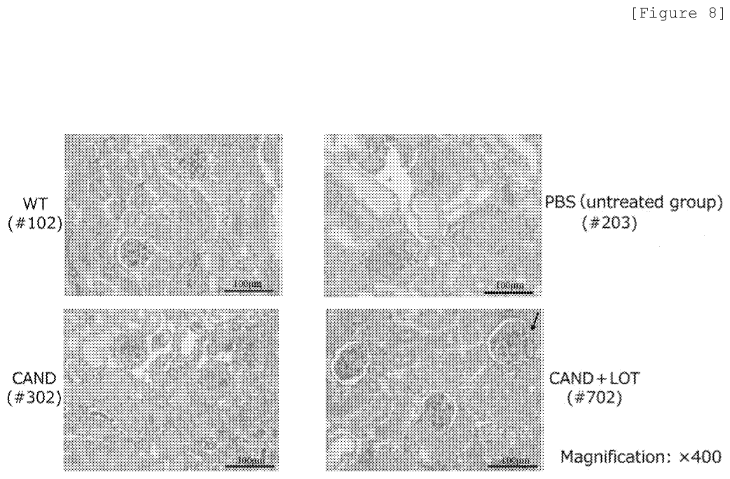

FIG. 8 shows the PAS-staining images of the kidneys in wild-type mice, the PBS group, the candesartan monotherapy group, and the candesartan and Lotriga combination therapy group.

In the PBS group and the candesartan monotherapy group, glomerulosclerosis, in which PAS-positive substances were deposited on the mesangial substrate, and growth of crescent cells were observed. In the PBS group, PAS-positive hyaline casts were also confirmed in an area of the renal tubular expansion. In the candesartan and Lotriga combination therapy group, glomerulosclerosis and crescent formation were rarely observed, and no change was observed in either the renal tubules or the interstitium. The arrow shows an example of the normal glomerulus.

### 8.9. Histopathological Test of the Kidneys (MT Staining)

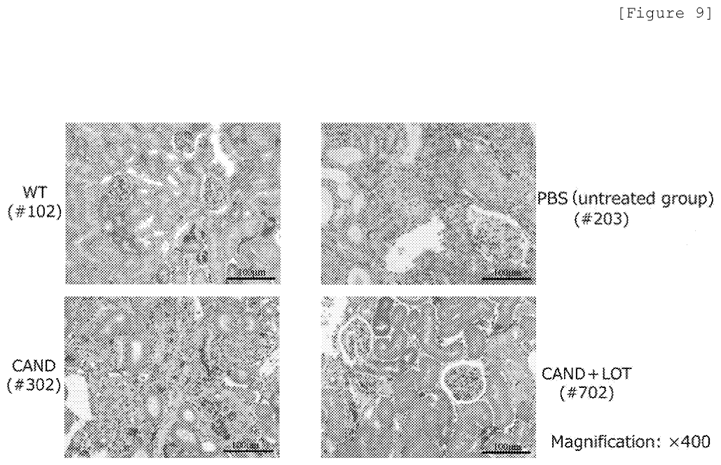

FIG. 9 shows the MT-staining images of the kidneys in wild-type mice, the PBS group, the candesartan monotherapy group, and the candesartan and Lotriga combination therapy group.

Fibrosis was observed in an area of crescent hyperplasia and in the renal tubular interstitium in the PBS group and the candesartan monotherapy group, but fibrosis was not observed in the candesartan and Lotriga combination therapy group.

### 8.10. Histopathological Test of the Kidneys (PAM Staining)

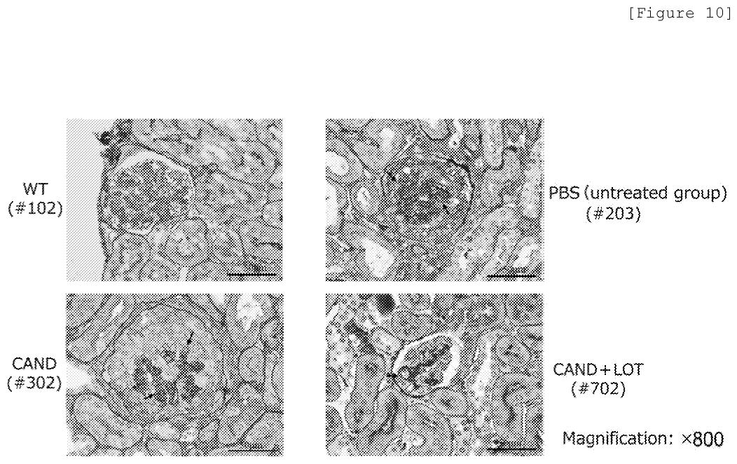

FIG. 10 shows the PAM-staining images of the kidneys in wild-type mice, the PBS group, the candesartan monotherapy group, and the candesartan and Lotriga combination therapy group.

A double contour of the loop wall was observed in the PBS group, the candesartan monotherapy group, and the candesartan and Lotriga combination therapy group. The arrow shows an example of the double contour of the loop wall.

### 8.11. Assessment of Glomerular Damage

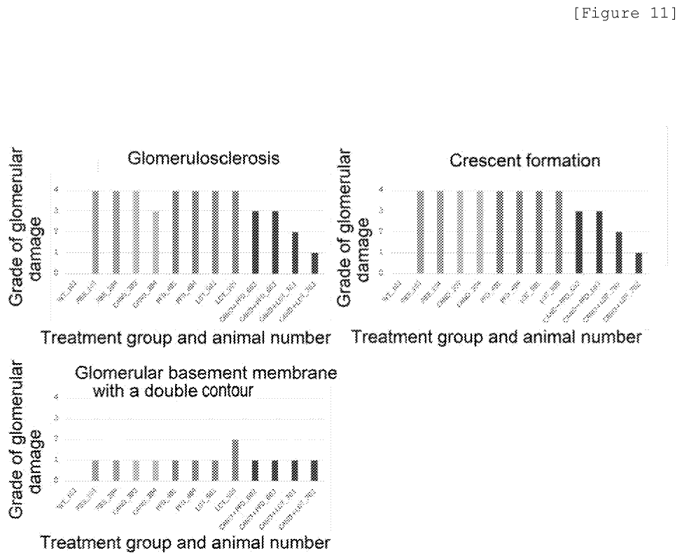

The severity of glomerular damage was assessed in each group of Alport syndrome model mice. Specifically, the severity was quantified for glomerulosclerosis, crescent formation, and basement membrane with a double contour with five levels (0, no change; 1, slight; 2, mild; 3, moderate; 4, severe. Refer to “Guidance on pathological diagnosis of diabetic nephropathy and hypertensive nephrosclerosis,” Tokyo Igakusha). FIG. 11 shows the results of this assessment.

Apart from wild-type mice, the severity of glomerular damage was lowest in the candesartan and Lotriga combination therapy group.

### 8.12. Assessment of Renal Tubular Damage

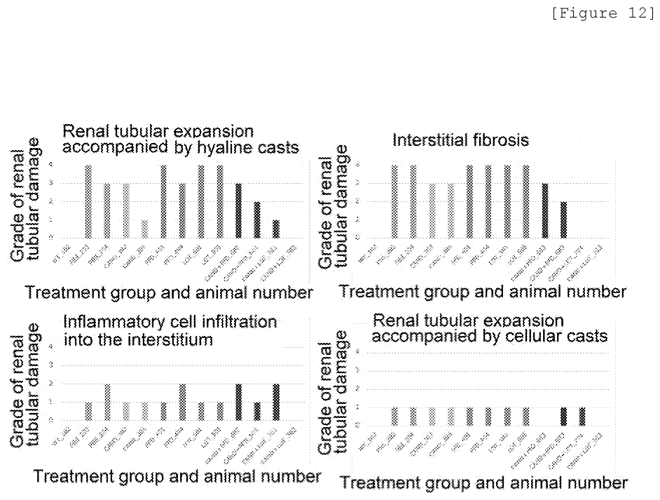

The severity of renal tubular damage was assessed in each group of Alport syndrome model mice. Specifically, the severity was quantified for renal tubular expansion accompanied by hyaline casts, interstitial fibrosis, inflammatory cell infiltration into the interstitium, and renal tubular expansion accompanied by cellular casts with five levels (0, no change; 1, slight; 2, mild; 3, moderate; 4, severe. Refer to “Guidance on pathological diagnosis of diabetic nephropathy and hypertensive nephrosclerosis,” Tokyo Igakusha). FIG. 12 shows the results of this assessment.

Apart from wild-type mice, the severity of renal tubular damage was lowest in the candesartan and Lotriga combination therapy group.

### Example 2

i. Subcontracting of the Study

The following study was subcontracted to an external facility.

### 2. Objective

An omega-3 fatty acid ethyl alone or in combination with a renin-angiotensin-system inhibitor is administered to Alport syndrome model (129-Col4a3tm 1Dec/J) mice, and the effect of suppressing progression of chronic renal failure is investigated.

### 3. Animal Welfare

All animal experiments performed in this subcontracted study were planned in accordance with the laboratory animal welfare rules of the subcontractor and performed after approval by the laboratory animal welfare committee. Of note, the conducted animal study complied with “Basic guidelines for the implementation of animal experiments etc. at research laboratories under jurisdiction of the Ministry of Agriculture, Forestry and Fisheries” (2006 Agriculture, Forestry and Fisheries Research Council Office Notification) and the like.

### 4. Study Materials

### 4.1. Test Substances (Known Compounds)

### 4.1.1. Names

TABLE 3

| | | General | |
|---|---|---|---|
| Generic name | Brand name | description | Storage condition |
| Candesartan | Candesartan | White powder | Refrigerated |
| cilexetil | | | (0° C.-10° C.) |
| Enalapril | Enalapril | White powder | Room temperature |
| maleate | | | (15° C.-25° C.) |
| Valsartan | Valsartan | White powder | Room temperature |
| | | | (15° C.-25° C.) |
| Omega-3 | Lotriga | Granular capsule | Room temperature |
| fatty acid | | (solution) | (15° C.-25° C.) |
| ethyl (1) | | | |
| Omega-3 | DHA97E | Colorless-light | Frozen(−20° C.) |
| fatty acid | | yellow solution | |
| ethyl (2) | | | |
| Omega-3 | EPA97E | Colorless-light | Frozen(−20° C.) |
| fatty acid | | yellow solution | |
| ethyl (3) | | | |

EPA97E and DHA97E which are prepared by the study subcontractor are to be used.

### 4.1.2. Method for Preparing Test Substances Etc.

Test substances were prepared for each use before use on the experiment day.

### Candesartan Cilexetil (Hereinafter Referred to as Candesartan: Tokyo Kasei Kogyo Co., Ltd.)

Candesartan was dissolved at a dose of 1.12 mg/mL in phosphate-buffered saline (hereinafter referred to as PBS) to prepare a dosing solution (2.8 mg/2.5 mL/kg) for Group Numbers (8), (9), and (10). A solution was prepared by diluting this dosing solution with an equal amount of PBS as a dosing solution (2.8 mg/5 mL/kg) for Group Number (7). Further, candesartan was dissolved at a dose of 4 mg/mL in PBS to prepare a dosing solution (10 mg/2.5 mL/kg) for Group Numbers (11) and (12).

### Enalapril Maleate (Hereinafter Referred to as Enalapril: Tokyo Kasei Kogyo Co., Ltd.)

Enalapril was dissolved at a dose of 3.28 mg/mL in PBS to prepare a dosing solution (8.2 mg/2.5 mL/kg) for Group Number (4). A solution was prepared by diluting this dosing solution with an equal amount of PBS as a dosing solution (8.2 mg/5 ml/kg) for Group Number (3).

### Valsartan (Tokyo Kasei Kogyo Co., Ltd.)

Valsartan was dissolved at a dose of 53.44 mg/mL in PBS to prepare a dosing solution (133.6 mg/2.5 mL/kg) for Group Number (6). A solution was prepared by diluting this dosing solution with an equal amount of PBS as a dosing solution (133.6 mg/5 ml/kg) for Group Number (5).

### Omega-3 Fatty Acid Ethyl (1) (Hereinafter Referred to as Lotriga: Takeda Pharmaceutical Co., Ltd.)

Lotriga was suspended in a 0.5% methylcellulose solution (hereinafter referred to as MC) to prepare a dosing solution at 1671.4 mg/2.5 mL/kg for Group Numbers (4) and (6) and a dosing solution at 501.0 mg/2.5 mL/kg for Group Number (8).

### Omega-3 Fatty Acid Ethyl (2) (Hereinafter Referred to as DHA97E: Bizen Chemical Co., Ltd.)

DHA97E was suspended in 0.5% MC to prepare a dosing solution at 421.0 mg/2.5 mL/kg for Group Number (9) and a dosing solution at 1503.6 mg/2.5 mL/kg for Group Number (11). A dosing solution was prepared by diluting this dosing solution with an equal amount of PBS as a dosing solution (1503.6 mg/5 ml/kg) for Group Number (13).

### Omega-3 Fatty Acid Ethyl (3) (Hereinafter Referred to as EPA97E: Bizen Chemical Co., Ltd.)

EPA97E was suspended in 0.5% MC to prepare a dosing solution at 421.0 mg/2.5 mL/kg for Group Number (10) and a dosing solution at 1503.6 mg/2.5 mL/kg for Group Number (12).

TABLE 4

| | Monotherapy | | | |
|---|---|---|---|---|
| | group | | | |
| | (Final | Combination therapy group | | |
| | concentration) | (4) A-LTH | (6) V-LTH | (8) C-LT |
| Enalapril | 8.2 mg/ | 8.2 mg/ | — | — |
| | 5 mL | 2.5 mL | | |
| Candesartan | 2.8 mg/ | — | — | 2.8 mg/ |
| | 5 mL | | | 2.5 mL |
| Valsartan | 133.6 mg/ | — | 133.6 mg/ | — |
| | 5 mL | | 2.5 mL | |
| Lotriga | — | — | — | 501.0 mg/ |
| | | | | 2.5 mL |
| Lotriga(high | — | 1671.4 mg/ | 1671.4 mg/ | — |
| dose) | | 2.5 mL | 2.5 mL | |

TABLE 5

| | Combination therapy group | | | |
|---|---|---|---|---|
| | (9)C-D | (10)C-E | (11)C-DH | (12)C-EH |
| Candesartan | 2.8 mg/ | 2.8 mg/ | 10 mg/ | 10 mg/ |
| | 2.5 mL | 2.5 mL | 2.5 mL | 2.5 mL |
| DHA97E | 421.0 mg/ | — | — | — |
| | 2.5 mL | | | |
| DHA97E(high dose) | — | — | 1503.6 mg/ | — |
| | | | 2.5 mL | |
| EPA97E | — | 421.0 mg/ | — | — |
| | | 2.5 mL | | |
| EPA97E(high dose) | — | — | — | 1503.6 mg/ |
| | | | | 2.5 mL |

### 4.2. Animal Experiment

### 4.2.1. Animals Used

Male 129-Col4a3tm 1Dec/J mice (hereinafter referred to as Alport syndrome (AS) mice) and wild type mice of the same strain (WT mice) were introduced into the study facility at three weeks old, and healthy animals (four weeks old), which did not show any abnormality in general signs during the acclimatization period, were subjected to the experiment.

### 4.2.2. Breeding Environment

Up to five animals were housed in a breeding cage (189 mm×298 mm×128 mm) and bred in an animal breeding room adjusted to the following environment: temperature, 20° C. to 25° C.; humidity, 40% to 70%; ventilation rate, 10 times or more/hour; light on, 12 hours (7:00 to 19:00). Animals were given solid feed CRF-1 (Oriental Yeast Co., Ltd.) ad libitum. As a drinking water, water filtered with a filter was given ad libitum.

### 5. Experimental Methods

### 5.1. Group Composition

TABLE 6

| | No. of | Group | Abbreviated | Substance | Dose | Volume administered |
|---|---|---|---|---|---|---|
| Mouse | animals | Number | group name | administered | (mg/kg) | (mL/kg) |
| WT | 5 | (1) | W | PBS | — | 5.0 |
| AS | 5 | (2) | P | PBS | — | 5.0 |
| AS | 5 | (3) | A | Enalapril | 8.2 | 5.0 |
| AS | 5 | (4) | A-LTH | Enalapril | 8.2 | 2.5 |
| | | | | Lotriga (high dose) | 1671.4 | 2.5 |
| AS | 5 | (5) | V | Valsartan | 133.6 | 5.0 |
| AS | 5 | (6) | V-LTH | Valsartan | 133.6 | 2.5 |
| | | | | Lotriga (high dose) | 1671.4 | 2.5 |
| AS | 5 | (7) | C | Candesartan | 2.8 | 5.0 |
| AS | 5 | (8) | C-LT | Candesartan | 2.8 | 2.5 |
| | | | | Lotriga | 501.0 | 2.5 |
| AS | 5 | (9) | C-D | Candesartan | 2.8 | 2.5 |
| | | | | DHA97E | 421.0 | 2.5 |
| AS | 5 | (10) | C-E | Candesartan | 2.8 | 2.5 |
| | | | | EPA97E | 421.0 | 2.5 |
| AS | 5 | (11) | C-DH | Candesartan | 10.0 | 2.5 |
| | | | | DHA97E | 1503.6 | 2.5 |
| AS | 5 | (12) | C-EH | Candesartan | 10.0 | 2.5 |
| | | | | EPA97E | 1503.6 | 2.5 |
| AS | 2 | (13) | DH | DHA97E | 1503.6 | 5.0 |

### 5.2. Grouping

AS mice identified as Homo mice by a genotyping test were grouped into Group Numbers (2) to (13) on the basis of the body weight at four weeks old, so that the mean body weight should be close between groups. Five animals were extracted from Group Number (1), so that body weight should be close to those of the AS mice groups. After grouping, individual animals were identified using ear tags. Animals excluded from the study and animals found to have abnormality during the acclimatization period were euthanized.

### 5.3. Observation of General Signs

General signs were observed once daily from the experiment start day (grouping day) to the experiment completion day (sampling day).

### 5.4. Measurement of Body Weight

Body weight was measured using a scale for animals twice weekly from the grouping day and daily from the day of the first animal death until the sampling day of each group.

### 5.5. Administration of Test Substances

Animals were restrained, and a test substance was administered directly into the stomach using a 1-mL syringe and a sonde (Fuchigami Kikai, CAT No. 5200). Administration was performed in accordance with the conditions described in Section 5.1., so that a volume of 5 mL/kg should be administered to each animal. The volume administered was calculated on the basis of the most recent (before the administration day) body weight.

### 5.6. Collection of Biomaterials

### 5.6.1. Collection Day

At the timepoint when all animals in Group Number (2) were euthanized or died, the biomaterial collection day was decided after discussion with the study subcontractor.

### 5.6.2. Collecting Methods

Death was confirmed by visually observing cardiopulmonary arrest (euthanasia). Then, the left and right kidneys, the left and right femurs, the left and right eyeballs, the cochlea, the heart, the liver, and the pancreas were collected and trimmed, and the wet weights of the left and right kidneys, the left and right femurs, and the heart were measured.

Among the collected biotissues, the left and right femurs were immersed in a solution of 70% ethanol (Fujifilm Wako Pure Chemical Corporation). Other tissues were immersed in a 10% neutral buffered formalin solution (Fujifilm Wako Pure Chemical Corporation). Of note, the right kidney was divided into two at the renal pelvis and immersed after to obtain kidney specimens.

### 6. Data Analysis

The mean and standard error of the body weight and the wet weight of each organ were calculated for each study group. Further, each of the measured values was compared between the Group Number (2) and each of other groups. A statistical analysis add-in software “Excel Statistics 2015” (Social Survey Research Information Co., Ltd.) was used for statistical processing, and a significant difference was determined with a hazard ratio (p value)<0.05.

### 7. Humanitarian Endpoint

Euthanasia treatment was performed for animals excluded from the study as a result of grouping or animals which developed such aggravated conditions as listed below or a refractory condition. Euthanasia treatment was performed by a carbon dioxide inhalation method.
Rapid body weight loss: loss of 25% or more in seven days
Distress symptoms: dyspnea, piloerection, crouching position, extreme cyanosis
Long-term abnormalities in appearance with no sign of recovering: inability to change the body position, diarrhea, hemorrhage, vulvar stains

### 8. Experimental Results

8.1. Changes with Time in the Survival Rate

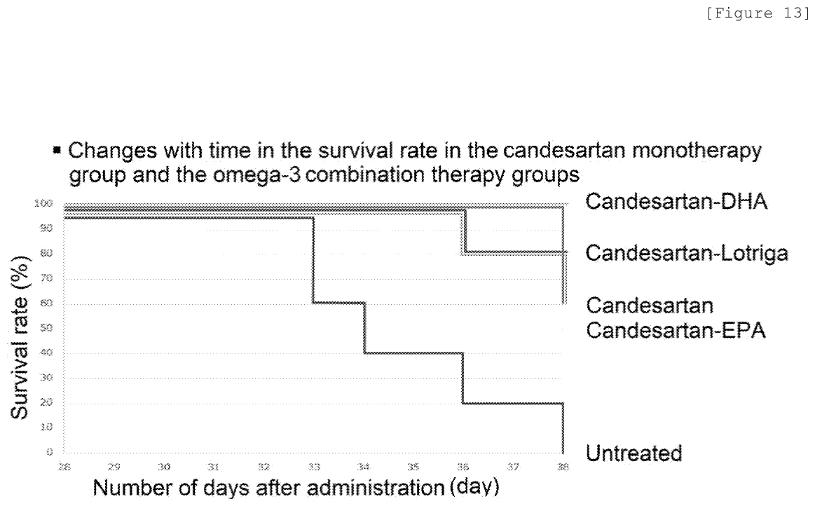

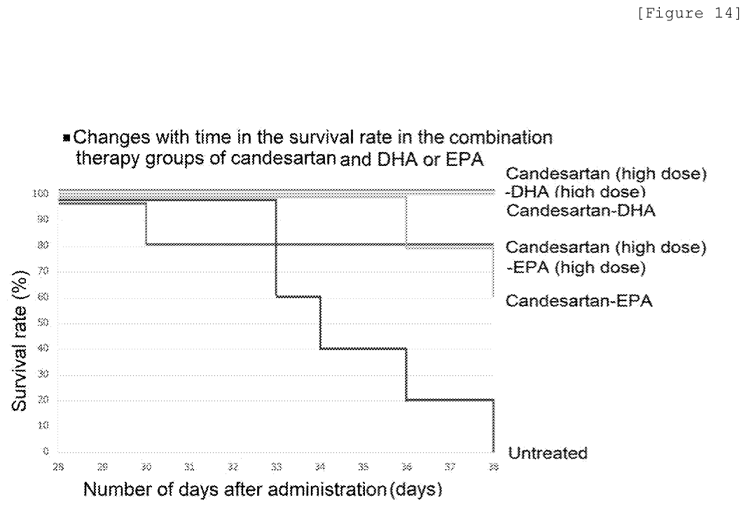

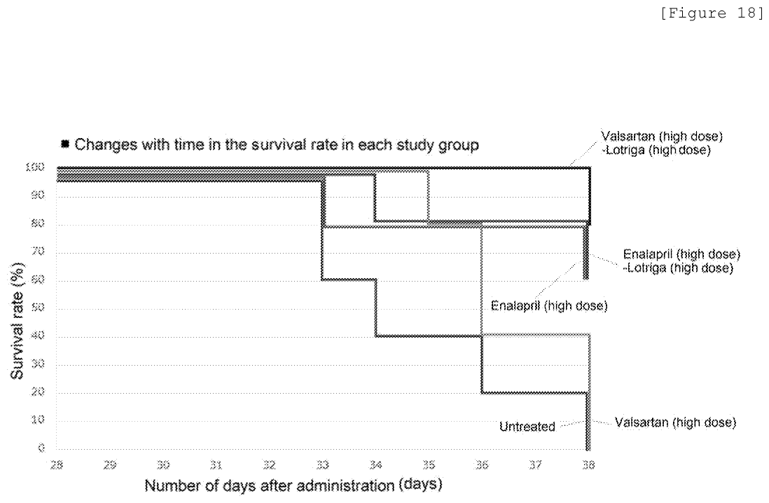

FIGS. 13, 14, and 18 show changes with time in the survival rate in each study group. As shown in FIG. 13, the use of candesartan in combination with a DHA ethyl ester showed a higher survival rate than the use in combination with Lotriga, indicating that a DHA ethyl ester can be a therapeutic agent for Alport syndrome. However, the use in combination with an EPA ethyl ester showed no difference from candesartan monotherapy. As shown in FIG. 14, even a usual-dose DHA ethyl ester exhibited an effect higher than a high-dose EPA ethyl ester. As shown in FIG. 18, Lotriga also exhibited an effect when used in combination with valsartan, which is an active ARB, and the survival rate in the valsartan (high dose)-Lotriga (high dose) combination therapy group was substantially higher than that in the valsartan (high dose) monotherapy group. However, enalapril, which is an ACE inhibitor, did not show an effect resulting from the use in combination with Lotriga.

8.2. Changes with Time in the Mean Body Weight

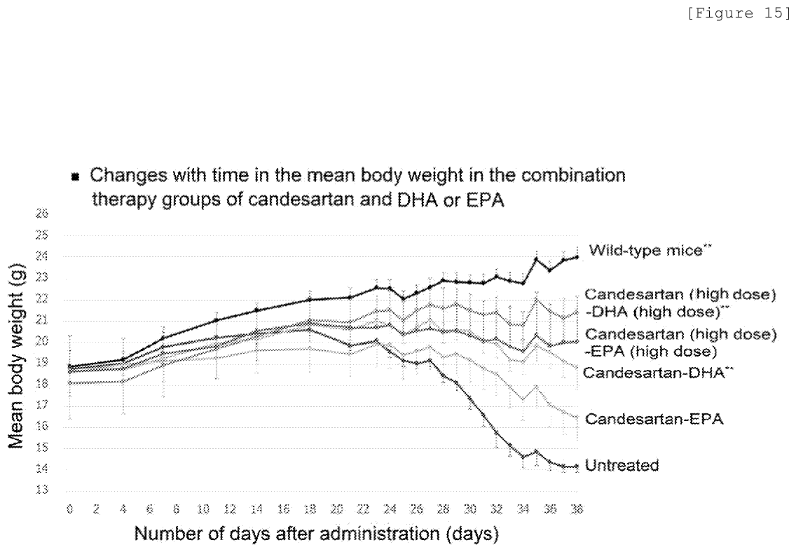

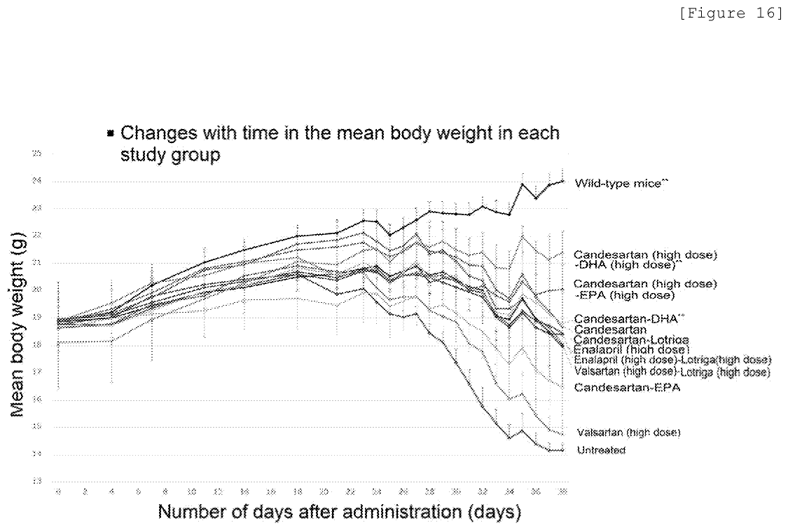

FIGS. 15 and 16 show changes with time in the mean body weight in each study group. As shown in FIG. 15, both a DHA ethyl ester and an EPA ethyl ester showed an effect of suppressing body weight loss when each was used in combination with candesartan. The effect of the DHA ethyl ester was much higher than that of the EPA ethyl ester, and the changes with time in body weight particularly in the high-dose groups was comparable with that in wild-type mice. As shown in FIG. 16, only the mean body weight in the groups receiving a DHA ethyl ester significantly increased as compared with the untreated group.

### 8.3. Relationship Between the Survival Rate and the Mean Body Weight

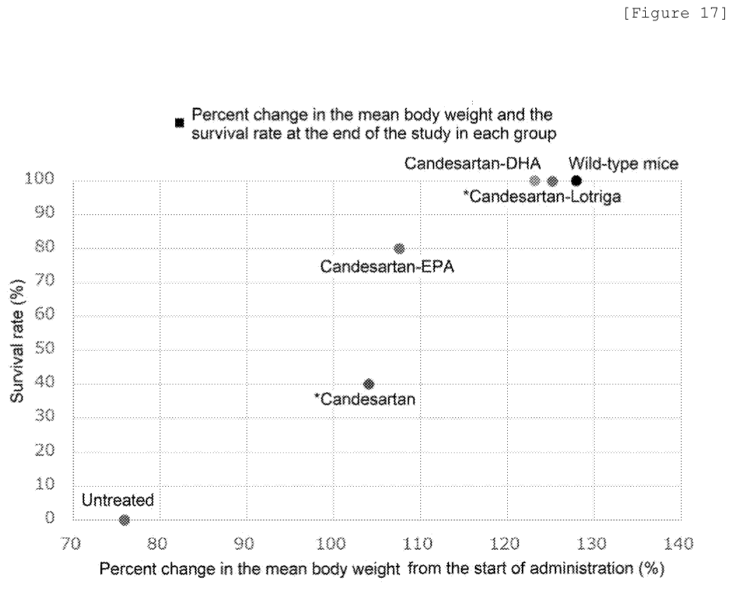

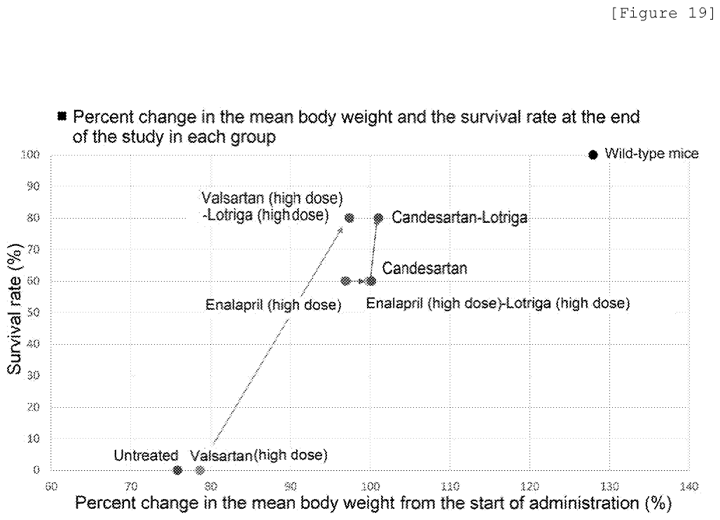

FIGS. 17 and 19 shows the percent change in the mean body weight and the survival rate in each group at the end of the study. FIG. 17 shows the study groups receiving candesartan dose 10 mg/kg/day and the control groups in Example 1 (*) and Example 2. As shown in FIG. 17, the survival rate in the candesartan-DHA ethyl ester combination therapy group was 100%, the percent change in the mean body weight was also comparable with that in wild-type mice, and it was demonstrated that a DHA ethyl ester had an excellent effect of suppressing progression of chronic renal failure. Further, as compared with candesartan monotherapy, an EPA ethyl ester also greatly improved the survival rate when used in combination with candesartan, confirming an excellent effect of suppressing progression of chronic renal failure. As shown in FIG. 19, not only candesartan, which is a prodrug, but also valsartan, which is an active form, improved the survival rate when used in combination with Lotriga, showing a high percent change in the body weight.

All publications, patents, and patent applications cited in the present specification are incorporated as they are into the present specification as reference.

### INDUSTRIAL APPLICABILITY

The present invention relates to medicines and therefore can be used in industries such as manufacture of medicinal products.

1. A method of treating a chronic kidney disease which comprise administering to a patient in need thereof a pharmaceutical composition comprising a) a renin-angiotensin-system inhibitor and b) at least one type of an omega-3 polyunsaturated fatty acid, an ester thereof, a metabolite thereof, a pharmaceutically acceptable salt thereof, or a triglyceride thereof.

2. The method according to claim 1, wherein the chronic kidney disease is a kidney disease with glomerular damage.

3. The method according to claim 1, wherein the chronic kidney disease is a kidney disease with podocyte damage or a kidney disease with glomerular basement membrane damage.

4. The method according to claim 1, wherein the chronic kidney disease is a tubular kidney disease or an interstitial kidney disease.

5. The method according to claim 1, wherein the chronic kidney disease is Alport syndrome.

6. The method according to claim 1, wherein the renin-angiotensin-system inhibitor is an angiotensin II receptor antagonist.

7. The method according to claim 1, wherein the renin-angiotensin-system inhibitor is candesartan cilexetil.

8. The method according to claim 1, wherein the renin-angiotensin-system inhibitor is valsartan.

9. The method according to claim 1, wherein the at least one type of an omega-3 polyunsaturated fatty acid is eicosapentaenoic acid, docosahexaenoic acid, or eicosapentaenoic acid and docosahexaenoic acid.

10. The method according to claim 1, wherein the at least one type of an omega-3 polyunsaturated fatty acid is eicosapentaenoic acid and docosahexaenoic acid.

11. The method according to claim 1, wherein the at least one type of an omega-3 polyunsaturated fatty acid is docosahexaenoic acid.

12. The method according to claim 1, wherein the at least one type of an omega-3 polyunsaturated fatty acid is eicosapentaenoic acid.

13. The method according to claim 1, wherein a) the renin-angiotensin system inhibitor and b) at least one type of the omega-3 polyunsaturated fatty acid, an ester thereof, a metabolite thereof, a pharmaceutically acceptable salt thereof, or a triglyceride thereof are administered to the patient at different times.
